(19) United States
(12) Patent Application Publication
(10) Pub. No.: US 2004/0186501 A1
(43) Pub. Date: Sep. 23, 2004

(54) Needle sheath for body/eyebrow tattoo machine

(76) Inventor: Chin Kuei Su, Taipei (TW)

Correspondence Address: Chin Kuei SU, PO Box 82-144, Taipei, TW

(21) Appl. No.: 10/392,939

(22) Filed: Mar. 21, 2003

(51) Int. Cl. A61B 17/34

(52) U.S. Cl. 606/185

(57) ABSTRACT

An improved structure of needle sheath for a body/eyebrow tattoo machine is comprised of an eyebrow tattoo machine and an eyebrow tattoo needle; the tip of the tattoo machine containing the needle sheath and the opening where the needle sheath is provided being made in a configuration of a right triangle for multiple standardized round needles in cluster or in row to be respectively arranged being tangent to one another or resting only on one side of the opening to maintain the tips of the needles stable in reciprocal motion.

### BACKGROUND OF THE INVENTION

(a) Field of the Invention

The present invention is related to an improved structure of a needle sheath adapted to a body or eyebrow tattoo machine, and more particularly, to one that is adapted to the tip of a body or eyebrow tattoo machine so to provide the optimal resting structure for the eyebrow tattoo needle.

(b) Description of the Prior Art

As illustrated in FIG. 1 of the accompanying drawings, an eyebrow tattoo machine of the prior art is generally comprised of a tattoo machine (11) and a tattoo needle (12). Wherein, a disposal sheath (111) is provided at the tip of tattoo machine (11) while the tattoo needle (12) is inserted into the tip (FIG. 2A) of a connection rod (112) inside the tattoo machine (11). An eccentric shaft mechanism inside the tattoo machine (11) linked to the connection rod (112) drives the tattoo needle (12) exercising reciprocal motion for the tattoo needle (12) when dipped with ink to sting against the human skin for a eyebrow tattoo. To facilitate the ink penetrating into the needle sheath, the tattoo needle (12) must maintain a proper spacing as illustrated in FIG. 2B since the tattoo needle (12) exercises reciprocal motion relatively to the needle sheath (111). Accordingly, the tattoo needle (12) operates without a reliable support, and the operating tattoo machine (11) is vulnerable to significant jitter and vibration to directly affect the stability of the tattoo needle (12) while stinging for an eyebrow tattoo, thus the tattoo results. Furthermore, the retracting tattoo needle (12) increases the pressure in positive direction inside the needle sheath (111) resulting in unexpected delivery of the ink; on the contrary, the extending tattoo needle (12) causes the ink to be sucked into the tattoo machine (11) to contaminate the mechanism inside the tattoo machine (11). The retracting and/or extending tattoo needle (12) are attributable to shorter service life of the tattoo machine (11) and the sources of spreading virus to endanger human health. For row needles in linear arrangement, other type of tattoo machine is required. Therefore, it is not economic to maintain at the same time various types of tattoo machines.

### SUMMARY OF THE INVENTION

The primary purpose of the present invention is to provide an improved structure of a needle sheath for a body/eyebrow tattoo machine for the needle to maintain stable during reciprocal motion. To achieve the purpose, an opening at the tip of the tattoo machine is made in the configuration of a right triangle for multiple standardized round needles in cluster or in row to respectively be tangent to one another or rest only on one side of the opening to maintain the tips of the needles stable in reciprocal motion.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a schematic view showing an eyebrow tattoo machine of the prior art.

FIG. 2A is a sectional view showing a local part of the prior art.

FIG. 2B is a top view showing a cluster of needles and a needle sheath in the eyebrow tattoo machine of the prior art.

FIG. 3 is a perspective view of a preferred embodiment of the present invention.

FIG. 3A is an enlarged view of a portion of FIG. 3.

FIG. 4 is a sectional view showing a local part of the preferred embodiment of the present invention.

FIGS. 5A and 5B are front views of a needle sheath of the preferred embodiment of the present invention.

FIG. 6A is a perspective view showing a local part of a needle sheath opening of another preferred embodiment of the present invention.

FIG. 6B is a front view of the needle sheath opening of another preferred embodiment of the present invention.

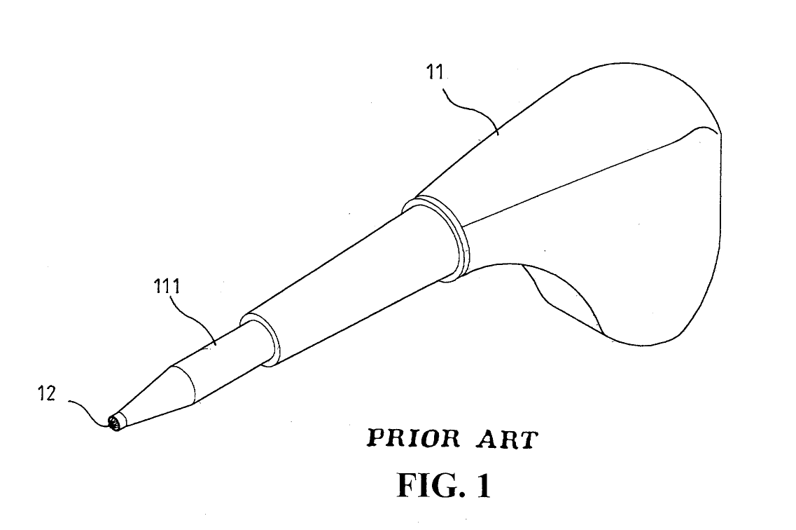

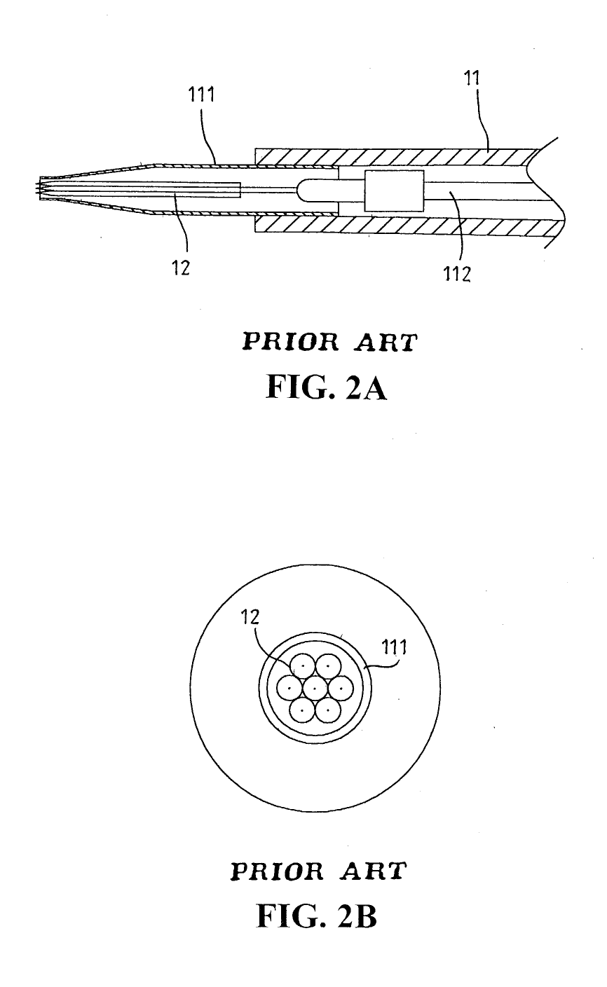

### DETAILED DESCRIPTION OF THE PREFERRED EMBODIMENT

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

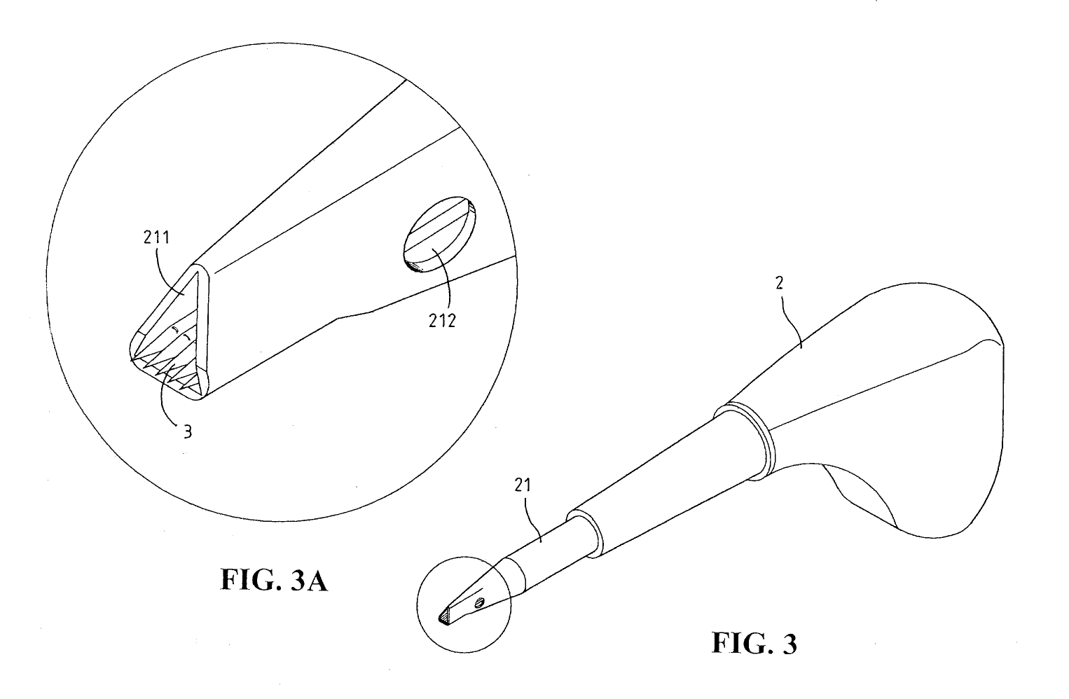

Referring to FIGS. 3 and 3A, an improved structure of a needle sheath for a body/eyebrow tattoo machine of the present invention is essentially comprised of an eyebrow tattoo machine (2) and multiple eyebrow tattoo needles (3). The tip of the eyebrow tattoo machine (2) contains a needle sheath (21) characterized by that the opening (211) of the needle sheath (21) is made in a configuration of a right triangle and is cut upward on its front an inclination not less than 45 degrees to give clear view for better observation as the eyebrow tattoo needles are approaching one's skin and to facilitate the sting process. One opening (212) or multiple openings (212) is provided on one side of the needle sheath (21) for ventilation to balance pressure inside and outside the needle sheath (21).

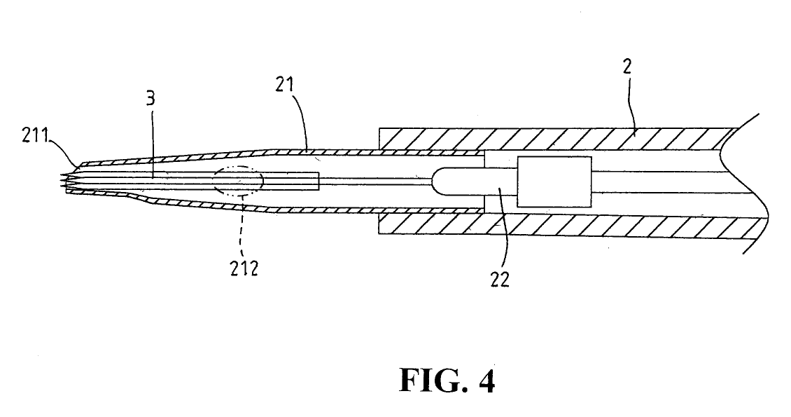

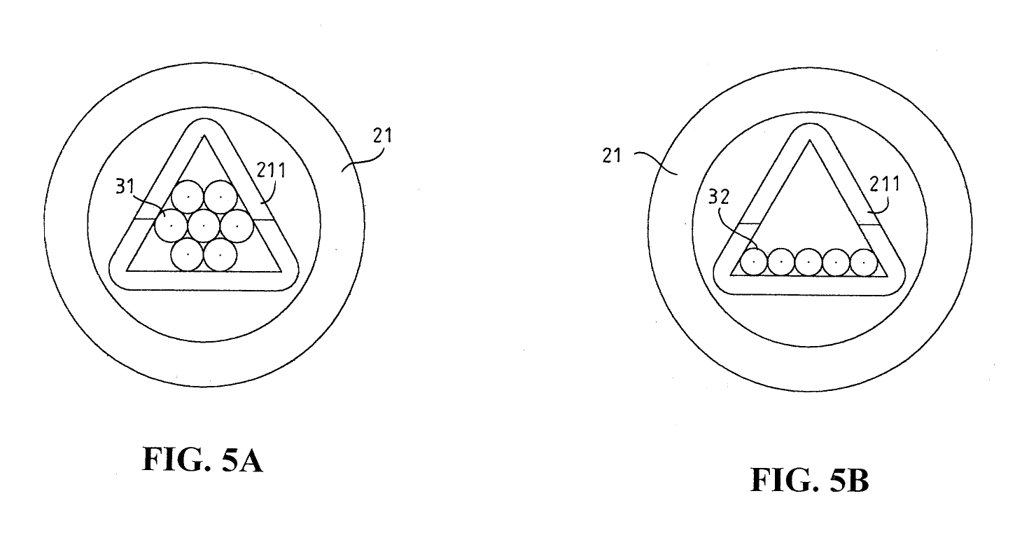

FIG. 4 shows the position of the eyebrow tattoo needle (3) when provided at the tip of a connection rod (22) in relation to the needle sheath (21). When a cluster of round needles is used, those needles are arranged in tangent to one another in the opening (212) of the needle sheath (21) as illustrated in FIG. 5A thus to be confined in a fixed position without jittering or producing noise while operating. Or alternatively, a row of needles is used, all the needles rest on the same side in the opening (211) of the needle sheath (21) as illustrated in FIG. 5B so that they are prevented from moving to either of the remaining two sides in the opening (211), thus to be kept their tips in stable position. Accordingly, a proper spacing is kept between those eyebrow tattoo needles and the opening (211) of the needle sheath (21), and the air pressure is balance due to the air ventilation (212) provided on the side of the needle sheath (21), ink flows smoothly without accidental spray.

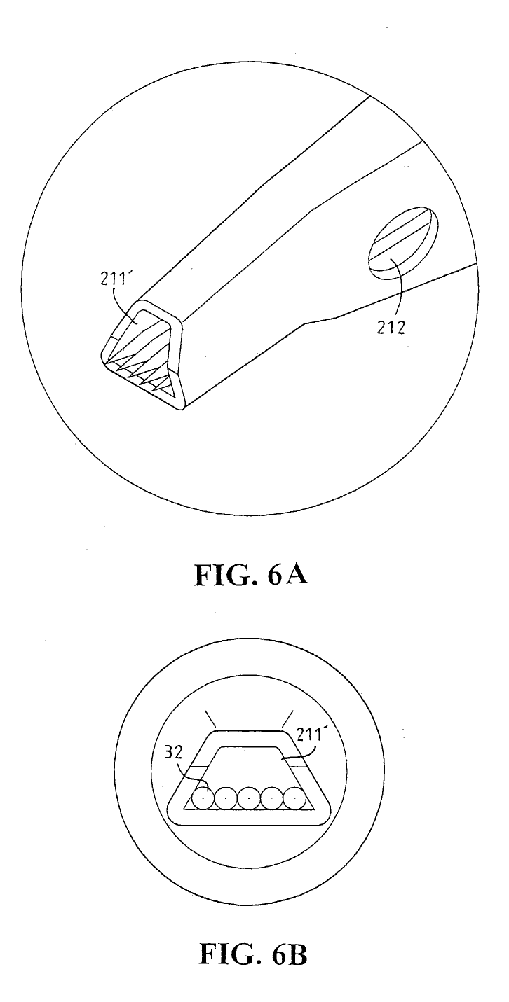

Now referring to FIGS. 6A and 6B for another preferred embodiment of the present invention, wherein, the upper part of the opening (211) of the needle sheath (21) otherwise provided in a right triangle configuration is cut into a trapezoid configuration to achieve the similar purpose of maintaining the tips of a row of needles in stable condition.

As disclosed above, the present invention offers the following advantages:

1. In the present invention, the opening of the needle sheath is made in a configuration of a right triangle for standardized round tattoo needles to be arranged either in tangent to one another or in a line resting only on one side to maintain the tips of those needles in stable while those needles exercise reciprocal motion, thus to upgrade stability and precision of eyebrow tattoo; and

2. The configuration of a right triangle provided for the opening of the needle sheath in the present invention allows it to be applicable to various types of eyebrow tattoo needles, thus to upgrade the value of the product and lower its production cost.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. An improved structure of needle sheath for a body/eyebrow tattoo machine comprising an eyebrow tattoo machine and an eyebrow tattoo needle; the tip of the tattoo machine containing the needle sheath and the opening where the needle sheath is provided being made in a configuration of a right triangle; the opening being cut upward for an angle not less than 45 degrees; multiple standardized round needles in cluster or in row being arranged respectively being tangent to one another or resting only on one side of the opening to maintain the tips of the needles stable in reciprocal motion.

2. An improved structure of needle sheath for a body/eyebrow tattoo machine as claimed in claim 1, wherein, the opening of the needle sheath is provided in a trapezoid configuration.
